(19) United States
(12) Patent Application Publication
(10) Pub. No.: US 2002/0110531 A1
(43) Pub. Date: Aug. 15, 2002

(54) Cholesterol reducing composition and method of making the same

(76) Inventors: Steven Faust, Oak Ridge, NJ (US); Jesse J. Kiefer, Belvidere, NJ (US); Anthony John Bell, Andover, NJ (US)

Correspondence Address: PFIZER, INC., 201 TABOR ROAD, MORRIS PLAINS, NJ, 07950, US

(21) Appl. No.: 09/783,257

(22) Filed: Feb. 14, 2001

(51) Int. Cl. A61K 9/68; A61K 31/56

(52) U.S. Cl. 424/58; 424/440; 514/178

(57) ABSTRACT

Stanol compound containing compositions containing a cholesterol reducing effective amount of at least one stanol compound which is an effective way of ingesting the stanol compounds to achieve the desired cholesterol reducing effect.

### FIELD OF THE INVENTION

The present invention is generally directed to a composition in the form of a soft chewy confection, hard boiled candy or chewing gum composition which contains a sufficient amount of at least one plant stanol so that the composition may be used as a means of reducing cholesterol in humans. The present invention enables ingestion of an effective amount of the plant stanol without having to ingest large quantities of the composition containing plant stanol or having to ingest a relatively large number of individual servings of such products.

### BACKGROUND OF THE INVENTION

Plant stanols and derivatives thereof (e.g. phytosterols and esters thereof) are known to possess cholesterol-reducing properties. These compounds are generally referred to hereinafter as stanol compounds. Such naturally occurring products have a chemical structure similar to that of cholesterol. They are considered essential constituents or properly functioning cells. Stanol compounds are typically C₂₆-C₃₀ alcohols which have an aliphatic chain in the C-17 position. Stanol compounds are believed to reduce cholesterol by binding to cholesterol and thereby forming a complex which is readily passed out of the body. Since high blood cholesterol levels have consistently been implicated as an important risk factor in cardiac and vascular diseases, the reduction of blood cholesterol is seen as a means of reducing cardiac and vascular disease.

Stanol compounds have been combined with various food products to provide a convenient means of administering the active agent to humans. There are currently two commercially available margarine based products known as Benecol (a trademark of Raisio Benecol Ltd, of Rasio Finland) and Take Control (a trademark of Lipton Company of Englewood Cliffs, N.J.), each of which contains a stanol compound. The stanol compound containing margarine is applied and used in a manner similar to conventional margarine products.

Other stanol compound-containing products are described, for example, in U.S. Pat. No. 5,591,836 which discloses the use of saponins bonded to sterols through a glycosidic linkage. The resulting complexes are used in a variety of products such as tablets, capsules, granules, cookies, wafers, candy products and the like for the purpose of reducing blood cholesterol levels.

WO99/15546 discloses the use of sistosterols as a dietary supplement in such food products as mustards, salad dressings, peanut butter, light spreads including margarine and mayonnaise as well as chocolate-flavored mint truffles.

WO98/58554 discloses the employment of a premix containing pulverized plant sterols and a conventional foodstuff raw material to prepare bakery products including bread, cake, pastries, crackers, biscuits, and the like.

European Patent Application No. 089767181 discloses the preparation of an aqueous dispersion of plant sterols and other high microlipids for use in spreads and other food products including beverages, dairy products, dry mixers, powdered non-dairy products, coffee, whiteners, milk shake mixes, confections, ice creams, instant milks, cake mixes, and the like.

Phytosterols have also been added to beverage compositions and acetic acid compositions such as disclosed in WO99/15547.

As indicated above, the incorporation of stanol compounds in wide variety of food products is known in the art. In many cases, the stanol compound is recognized as a cholesterol reducing agent. However, in order to obtain a cholesterol reducing effect in a human, the food product must be ingested in large quantities to provide a sufficient amount of the stanol compound so as to reduce and maintain cholesterol levels.

From a practical standpoint, the prior art food products contain relatively low levels of stanol compounds and therefore require the ingestion of large amounts of the food product to provide a sufficient amount of the stanol compounds to obtain a desired cholesterol reducing effect. There are two major disadvantages of such products. First, the amount of the food product which must be ingested is beyond the amount that the user is willing to ingest during the course of the day. For example, while stanol compounds have been added to spreads including margarine, typically the amount of margarine which must be consumed is far beyond what the average daily consumption is for a typical user.

A second disadvantage of typical food products containing stanol compounds, is that the food products must be ingested at least several times a day because individually the food products do not contain sufficient stanol compounds to obtain a meaningful cholesterol reducing effect. Under these circumstances, the typical user is not likely to follow a regimen which requires ingestion of several and up to many individual servings of a food product in order to obtain a cholesterol reducing effect. Such regimens have been problematical because they require discipline and often times the user does not have the food product available at appropriate times for consumption of multiple portions of the food product.

A still further disadvantage of typical food products containing stanol compounds is that they are not readily available to the user at all times during the course of the day. For example, the user may have a stanol compound containing margarine available only at home (i.e. it may not be available in restaurants or even when ingesting a meal on the go).

The key to obtaining a cholesterol reducing effect from food products containing stanol compounds is to have the food product in a readily available form, requiring a small number of individual servings, preferably only one or two servings, and which can be readily ingested. Of the previously mentioned food products known to contain plant stanols, none provide relatively easy and effective delivery of the plant stanols.

It would therefore be a significant advance in the art of reducing cholesterol if a product could be developed which contains a sufficient amount of stanol compounds so as to be an effective cholesterol reducing aid in the overall nutritional regimen of the intended user.

It would be a further advance in the art if a confectionery product, or gum composition could be produced which can reduce cholesterol and can be readily adapted to a users daily regimen over an extended period of time.

It would be a still further advance in the art if a confectionery product or gum composition could be developed which is effective in reducing cholesterol but does not require the user to ingest large volumes of a food product.

### SUMMARY OF THE INVENTION

The present invention is generally directed to a composition in the form of a soft chewy confection or hard boiled candy or a chewing gum composition which provides an effective amount of at least one stanol compound during the course of a dosage regimen which is readily facilitated by the user.

In a particular aspect of the present invention there is provided a soft chewy confection, hard boiled candy or chewing gum composition comprising a cholesterol reducing effective amount of at least one stanol compound.

In a further aspect of the present invention there is provided a soft chewy confection, hard boiled candy or chewing gum composition which delivers a cholesterol reducing effective amount of at least one stanol compound with a relatively small number of ingestable doses. In a preferred form of the present invention, the composition of the present invention includes a sufficient amount of the stanol compound such that an effective cholesterol reducing effect is obtained through the ingestion of no more than three doses of the composition per day, preferably only one or two doses per day.

In a more particular aspect of the present invention there is provided a soft chewy confection, hard boiled candy or chewing gum composition which provides from about 1.0 to 2.0 grams of at least one stanol compound per day, preferably in no more than three doses.

### DETAILED DESCRIPTION OF THE INVENTION

The present invention is directed to a particular class of compositions containing plant stanols which can be readily ingested by the user, employed in a relatively few doses and which is readily available. Applicants have determined that soft chewy confections, hard boiled candies and chewing gum compositions can be effectively provided with a sufficient amount of plant stanol that their customary use over the course of a typical day can deliver a cholesterol reducing effective amount to the user.

As used herein the term “stanol compound” shall mean any stanol compound regardless of origin such as a plant stanol which is capable of providing a cholesterol reducing effect. The amount of the stanol compound, through the ingestion of a relatively small number of doses of the confectionery product, is sufficient to provide a sustainable cholesterol reducing effect in the typical user. The plant stanol compound may be provided throughout the confectionery composition or in a portion thereof such as a centerfill portion.

Stanol compounds as used herein include plant stanols, sterols and sterol fatty acid esters and derivatives thereof which have a cholesterol reducing effect. The stanol compounds for use in the present invention are essential components of all plants. They are similar in function to that of cholesterol in mammals. The most abundant stanol compounds are beta-sitosterol, campesterol, and stigmasterol.

Stanol compounds have been known to effectively reduce serum cholesterol levels when administered in sufficient quantities. Even when administered in relatively small doses (a few grams a day) they reduce the absorbability of cholesterol and thus lower the serum total and LDL-cholesterol levels. It appears that stanol compounds displace cholesterol from the micellar phase and thereby prevent its absorption. One of the problems mitigating against using stanol compounds to effectively reduce cholesterol levels is that the absorption rate of stanol compounds is typically less than five percent of the amount consumed.

In accordance with the present invention, there is provided a composition in the form of a soft chewy confection, hard boiled candy or chewy gum composition which includes a sufficient amount of at least one stanol compound, preferably from about 1 to 2 grams per day, in a relatively small number of doses, typically no more than three doses per day, so that a cholesterol reducing effective amount of the active agent can be delivered to the user and the type of product and number of doses to reduce cholesterol are within a targeted regimen effective for at least most users.

As used herein the term “soft chewy confection” shall mean a soft confection readily chewable in the mouth such as fondants, caramels, toffees, fudge, marshmallows, soft nougats, jams, jellies and the like. Soft nougat compositions are especially suitable for the present invention.

The present invention encompasses plant sterols which are capable of delivering a cholesterol reducing effect and maintaining the cholesterol reducing effect when delivered in a confectionery product in accordance with the present invention.

As previously indicated, stanol compounds include sterols appearing in the plant kingdom which closely resemble the chemical structure of cholesterol. The sterols found in animals, plants and mushrooms of marine organisms and sea-weeds form a wide variety of oxidation, double bond, methyl group substitution and C-17 side-group structures. Using a catalyst, phytosterols isolated in commercial applications can be hydrogenated into corresponding stanols. The most well known phytosterols are beta-sitosterol, stigmasterol, campesterol, brassicasterol, cycloartenol, and cyclobranol. The sources of stanol compounds in human diet include plant oils and margarines made from plant oils, while phytosterols can be found in grain products, soybeans and rice.

In accordance with the present invention, the stanol compounds which can be used in the products of the present invention are those which have a cholesterol reducing effect and which can be placed in relatively large quantities in such products without detracting from the formation or taste of the product.

In accordance with the present invention, the plant stanols employed herein are combined with the ingredients of the products of the present invention in such an amount as to provide a cholesterol reducing effective amount of the stanol compound, preferably from about one to two grams per dose. The ingestion of one or two grams of stanol compounds results in a cholesterol reducing effect which can be maintained when the dosage regimen is maintained by the user. Products for use in the present invention are those which can effectively include a meaningful dose of stanol compounds so that the user can ingest a relatively few, preferably no more than three, portions of the product to achieve and maintain a cholesterol reducing effect. In addition, the products are desirably easy to ingest and easy to have at hand so that the user does not have to make special arrangements to take the desired amount of the stanol compound.

The products of the present invention are uniquely suited to provide the necessary daily amount of plant stanol within no more than 3 doses or servings per day. In addition, the present products are convenient to ingest at any time in any location. The products of the present invention include soft chewy confections, hard boiled candies and chewing gum compositions. The ingredients which can be used to formulate such products are generally well known.

The preparation of the present products is historically well known and has changed little through the years. Hard boiled candies, may be processed and formulated by conventional means. In general a hard boiled candy has a base composed of a mixture of sugar and other carbohydrate bulking agents kept in an amorphous or glassy condition, preferably having from about 0.5% to about 5.0% moisture, preferably from about 0.1% to about 4% moisture. The base normally contains up to about 90% sugar and up to 60% glucose syrup (e.g., corn syrup). Further ingredients such as flavoring agents, sweetening agents, acidulants, colorants and so forth may also be added. Hard boiled candies may also be prepared from non-fermentable sugars such as sorbitol, mannitol, xylitol, maltitol, isomalt, erythritol, hydrogentated starch hydrolysates and the like.

Such compositions may be routinely prepared by conventional methods such as those involving fire cookers, vacuum cookers, and scraped-surface cookers also referred to as high speed atmospheric cookers. Once the candy mass has been properly tempered, it may be cut into workable portions or formed into desired shapes. A variety of forming techniques may be utilized depending upon the shape and size of the final product desired. A general discussion of the composition and preparation of hard boiled candies may be found in E. B. Jackson, Ed. “Sugar Confectionery Manufacture”, 2^{nd} edition, Blackie Acadmic & Professional Press, Glasgow UK, (1990), at pages 129-169, incorporated herein by reference.

Soft chewy confectionery products, soft candy confection may be utilized in the present invention. Such products as previously indicated include fondants, caramels, toffees, fudge, marshmallows and nougats and the like and may also include jams and jellies. The preparation of soft chewy confectionery products, such as soft nougats, involves conventional methods as generally disclosed in E. B. Jackson “Sugar Confectionery Manufacturer”, 2^{nd} Edition mentioned above at pages 161-167. Such methods include the combination of two primary components, namely (1) a high boiling syrup such as a glucose syrup, or the like, and (2) a relatively light textured frappe, generally prepared from egg albumin, gelatin, vegetable proteins, such as soy derived compounds, sugarless milk derived compounds such as milk proteins, and mixtures thereof. Further ingredients such as flavoring agents, flavoring agenta, additional carbohydrate bulking agents, colorants, preservatives, medicaments, mixtures thereof and the like may be added thereafter also under agitation.

Preferred soft chewy compositions are soft nougats which are a combination of whipped candy and soft candy. Proteins such as egg whites or egg albumen are employed in which much of the moisture is removed while the egg whites are mixed with the other ingredients including honey, sugar, nuts such as pistachio and/or almonds and flavorings.

The chewiness of the batch of nougat is controlled by the percentage of non-crystalline sugars, such as corn syrup, invert sugar or honey that the batch contains, and the mixing method employed in manufacture.

The preparation of soft nougats is disclosed in “Choice Confections”, Waller Richmond (Chapter 14), pp. 251-267, Manufacturing Confectionery Publishing Company (1954), incorporated herein by reference.

The plant stanol compound may be added to the nougat composition at any time during preparation such as in combination with the protein which is then added to the other ingredients as previously described.

### EXAMPLE 1

### Preparation Of Boiled Candy Containing Plant Stanol

500 g of corn syrup, 500 g of sugar, 246.27 g of water, 20 g of fat and 145 g of plant stanol were combined to form a slurry. The slurry was cooked to approximately 145° C. or until the moisture in the final product was reduced to under 4%.

2 g of a colorant, 8 g of a flavorant and 5 g of citric acid were then added to the slurry. The molten mass was mixed in a cooker fitted with a scrap or swept surface blade or coil. The mass was tempered and cooled and formed into 5 g pieces of individual servings of the boiled candy. Each piece of boiled candy contained approximately 0.67 g of plant stanol.

### EXAMPLE 2

### Preparation Of Chewy Candy With Plant Stanol

783 g of corn syrup, 218 g of sugar, 107.37 g of water, 12 g of sorbitan monostearate and 4.5 g of monodiglycerides as emulsifiers and 300 g of plant stanol were combined to form a slurry. The slurry was cooked to approximately 125° C. or until the moisture of the final product was reduced to a range of from about 7 to 10%. 5 g of colorant, 40 g of flavorant and 25 g of citric acid are then added to slurry. The mass is allowed to cool while a mixture of 18.8 g of gelatin (175 bloom), 4.5 g of gum arabic and 32.5 g of water are added to the slurry while the same is mixed in a cooker equipped with a scrap or swept surface blade or coil. A resulting mixture was maintained at a temperature of approximately 100° C. under continuous mixing. Thereafter, the resulting mixture is cooled and when the mass reaches a temperature of approximately 40° C., 150 g of a fondant is added for seeding. The resulting mass is then formed and cut into 5 g pieces of chewy candy with each piece containing approximately 1 g of plant stanol. A cholesterol reducing effect is obtained by ingestion of two pieces of the chewable candy per day.

### EXAMPLE 3

### Preparation Of Fudge Candy Containing Plant Stanol

280 g of corn syrup, 76 g of sugar, 37.43 g of fat, 174 g of sweetened condensed whole milk, 2 g of enzyme modified soy protein, 0.49 g of lecithin, 1 g each of ammonium bicarbonate and salt and 170 g of plant stanol were combined in a cooker fitted with a scrap or swept surface blade or coil. The slurry was cooked to approximately 140° C. or until the moisture content of the final product was in the range of from about 7 to 10%.

38.67 g of flavorant were then added. The mass is allowed to cool under mixing to approximately 40° C. at which time 200 g of a fondant for seeding was added to the mixture. The mass was then formed into 5 g pieces of the fudge candy with each piece containing approximately 1 g of plant stanol. 2 pieces of the fudge candy provide a cholesterol reducing effective amount of the plant stanol to the average person.

### EXAMPLE 4

### Preparation of Chewing Gum Compositions With Plant Stanol

Gum cores containing a gum base composition containing butyl rubber, polyisobutylene, PVA (Polyvinylacetate), polyethylene, filler, and elastomer plasticizer were combined with a sufficient amount of plant stanol to provide from 1 to 2 g of plant stanol per piece. The gum cores were placed into a coating pan and broken into individual pieces. A sugarless solution containing 70% by weight of maltitol, as well as titanium dioxide, gum arabic and water was heated to between 70 and 80° C. The solution was sprayed on to the gum core pieces in layers and allowed to dry between sprays while the coating pan was continually rotating to ensure a smooth even coat of the gum cores.

The coating was built up to about 8% by weight of the final pellet weight. Ace K was then added and then covered with another layer of the above-mentioned coating solution and then allowed to dry.

### EXAMPLE 5

### Preparation of Chewing Gum Compositions With Plant Stanol

A gum base as described in Example 4, was combined with a sufficient amount of plant stanol to provide from 1 to 2 g of plant stanol per piece of gum to produce an uncoated chewing gum composition containing an effective amount of plant stanol.

What is claim is:

1. A stanol compound containing composition selected from the group consisting soft chewy confections, hard boiled candies and chewing gum compositions comprising a cholesterol reducing effect amount of at least one stanol compound.

2. The stanol compound containing composition of claim 1 in which the composition is selected from the group consisting of fondants, caramels, toffees, fudge, marshmallows and nougats.

3. The stanol compound containing confectionery composition of claim 2 wherein the composition is a nougat.

4. The stanol compound containing composition of claim 1 wherein the composition is a hard boiled candy.

5. The stanol compound containing composition of claim 1 wherein the composition is a chewing gum composition.

6. The stanol compound containing composition of claim 1 wherein the cholesterol reducing effective amount is from about 1 to 2 grams per day.

7. The stanol compound containing composition of claim 6 wherein no more than three serving portions of the composition together contain said cholesterol reducing effective amount of plant stanol.

8. The stanol compound containing composition of claim 7 wherein each serving of said compositions from about 1.0 to 2.0 g of plant stanol.

9. The stanol compound containing composition of claim 1 wherein the stanol compound is a plant stanol.

10. The stanol compound containing composition of claim 9 wherein the plant stanol compound is selected from the group consisting of beta-sitosterol, campesterol, and stigmasterol.

11. A method of forming stanol compound containing composition having a cholesterol reducing effect amount of at least one stanol compound, said method comprising combining said stanol compound with a slurry containing at least one ingredient of said composition to form a mixture, heating the mixture while stirring and cooling the mixture.

12. A method of reducing the amount of cholesterol in a warm blooded animal comprising providing the warm blooded animal with an effective amount of the composition of claim 1.
